(19) United States
(12) Patent Application Publication
(10) Pub. No.: US 2022/0001068 A1
(43) Pub. Date: Jan. 6, 2022

(54) SELF-DECONTAMINATING PERSONAL PROTECTIVE EQUIPMENT USING UV LIGHT

(71) Applicant: Accenture Global Solutions Limited, Dublin (IE)

(72) Inventors: Mark Benjamin Greenspan, San Francisco, CA (US); Aditi Maheshwari, San Francisco, CA (US)

(21) Appl. No.: 17/360,159

(22) Filed: Jun. 28, 2021

Related U.S. Application Data
(60) Provisional application No. 63/047,379, filed on Jul. 2, 2020.

(51) Int. Cl. A61L 9/20; A62B 18/08; A62B 23/02; A41D 13/11

(52) U.S. Cl. CPC A61L 9/20; A62B 18/08; A61L 2209/14; A41D 13/1107; A61L 2209/12; A62B 23/025

(57) ABSTRACT

This document describes personal protective devices. In one aspect, a personal protective device includes a face cover arranged to cover at least a nose and mouth of a person. The face cover includes one or more airflow channels that provide a path for air to flow between an exterior side of the face cover and an internal side of the face cover, and one or more ultraviolet (UV) light sources arranged to emit UV light into the one or more airflow channels.

### CROSS-REFERENCE TO RELATED APPLICATIONS

This application claims the benefit of U.S. Patent Application Ser. No. 63/047,379, filed Jul. 2, 2020, which is incorporated herein by reference.

### FIELD

This specification generally relates to self-decontaminating personal protective equipment.

### BACKGROUND

Face masks, such as surgical masks or cloth masks, and respirators are examples of personal protective equipment worn by people for protection against airborne particles and liquids reaching their faces. These masks and respirators typically include one or more layers of material that fit around a person's face and filter particles from reaching the person's respiratory tract and/or from escaping from the person into the air around them. These masks and respirators are typically designed to only be worn once, unless decontaminated for subsequent uses.

### SUMMARY

This specification generally describes self-cleaning personal protective equipment (PPE) that uses ultraviolet (UV) light to inactivate (e.g., kill) airborne viruses, germs, and other pathogens.

According to some implementations, a personal protective device can include a face cover arranged to cover at least the nose and mouth of a person. The face cover can include one or more airflow channels that provide a path for air to flow between an exterior side of the face cover and an internal side of the face cover. The face cover has one or more ultraviolet (UV) light sources arranged to emit UV light into the one or more airflow channels.

Implementations may include one or more of the following features. The one or more UV light sources can include one or more UV light emitting diodes (LEDs). The one or more UV light sources can be configured to emit far-UVC light having a wavelength of about 207 to 222 nm. The one or more light sources can include one or more UV LEDs that emit UV light through fiber optic hair-like fibers within the airflow channel(s).

At least one UV light source can be disposed within a given airflow channel and arranged to emit UV light opposite a direction of airflow within the given airflow channel. At least one UV light source can be disposed within a given airflow channel and arranged to emit UV light in a same direction of airflow within the given airflow channel. At least one UV light source can be arranged to emit UV light perpendicular to the direction of airflow within the airflow channel or at an angle to the direction of air flow, e.g., at a 45 degree angle, a 60 degree angle, or 130 degree angle, or another appropriate angle other than zero, 90, or 180 degrees.

The personal protective device can include a filter disposed on at least one side of the at least one UV light source. The at least one side can be either in front of the at least one UV light source such that the at least one UV light source emits UV light onto the filter or behind the at least one UV light source such that the at least one UV light source emits UV light away from the filter.

At least one airflow channel can include multiple UV light sources disposed about an inner perimeter of the at least one airflow channel and arranged to emit UV light into the at least one airflow channel. At least one airflow channel can include a clear area in a portion of a wall of the at least one airflow channel and a UV light source can be arranged to emit UV light through the clear area and into the airflow channel.

The face cover can include one or more layers of material that prevent air from flowing between the exterior side of the face cover and the internal side of the face cover without going through one of the one or more airflow channels. The face cover can include a respirator having an inhalation element and an exhalation element. The one or more airflow channels can be disposed within at least one of the inhalation element or the exhalation element.

At least one airflow channel can have a meandering airflow path between the exterior side of the face cover and the internal side of the face cover. The one or more airflow channels can be integrally formed with the face cover.

The one or more UV light sources can be arranged within the face cover such that, when the face cover is worn by the person, the UV light emitted by the UV light sources decontaminates air in the airflow channel and kills pathogens in the airflow channel before the air is inhaled by the person wearing the face cover.

According to another implementation, a method for manufacturing a personal protective device can include obtaining a face cover, installing one or more air flow channels within the face cover, and disposing one or more UV light sources in the face cover.

Installing one or more airflow channels within the face cover can include forming the one or more airflow channels integrally with the face cover. Installing one or more airflow channels within the face cover can include attaching a separate element to the face cover that forms the one or more airflow channels.

Disposing one or more UV light sources in the face cover can include arranging the one or more UV light sources inside the one or more airflow channels. Disposing one or more UV light sources in the face cover can include arranging the one or more UV light sources externally to the one or more airflow channels. Disposing one or more UV light sources in the face cover can include arranging the one or more UV light sources relative to a separate element. The one or more UV light sources can be arranged within the separate element.

The methods in accordance with the present disclosure can include any combination of the aspects and features described herein. That is, methods in accordance with the present disclosure are not limited to the combinations of aspects and features specifically described herein, but also may include any combination of the aspects and features provided.

The subject matter described in this specification can be implemented in particular embodiments so as to realize one or more of the following advantages.

The personal protective devices described in this document include UV light sources that emit UV light into airflow channels to inactivate or kill pathogens that enter the airflow channels, without harming the person wearing the personal protective device or any other person in close proximity. In this way, the personal protective devices are capable of killing airborne pathogens as soon as they enter the devices, increasing the efficacy of the devices against airborne diseases. The personal protective devices are also capable of killing airborne pathogens as soon as the user exhales into the devices. Creating a new filtering technique that does not rely on the multi-layer composition of conventional respirators (e.g., N95 respirators), provides an alternative form of respirator to help prevent any shortages of face masks, respirators, or other PPE in the event of a crisis and also reduces medical waste that arises due to the disposal of non-reusable PPE. The personal protective devices described in this document also eliminate or prevent cross-contamination from infected PPEs that are worn multiple times.

Low doses of far-UVC radiation, e.g., 207-222 nanometer (nm) can be used to inactivate or effectively kill pathogens without damaging a person's skin or other tissue even if the far-UVC light reaches the person's skin. Other implementations may use wavelengths outside the far-UVC range in combination with one or more layers of UV-light blocking material.

In some implementations, the airflow channel can include a meandering airflow path. The meandering path can allow the airflow channel to be longer without further increasing the thickness of the face cover at the location of the airflow channel. That is, the meandering path can reduce any protrusion of the face cover required to fit the airflow channel within the face cover, making the face cover less bulky. The meandering path can also enable more UV light elements to be disposed in the airflow channel or for a longer decontamination path for the air. The meandering path also enables the UV light elements to be arranged such that the UV light sources are not directed at the wearer's skin. For example, the UV light elements can be disposed in vertical portions of the airflow channel such that the UV light sources of the UV light elements are not directed at the wearer. In another example, the UV light sources can be arranged in non-vertical channels, but directed away from the interior side of the face covering that would be adjacent to the wearer's face when the face covering is worn.

The UV light source may be arranged to emit UV light into the airflow channel through a clear area formed along a perimeter of the airflow channel. This configuration may allow the UV light source to be mounted externally to the airflow channel while still emitting UV light into the airflow channel. This can reduce the drag on the flow of air caused by UV light elements disposed in the airflow channel. These and other implementations of the present disclosure may improve the breathability and comfort of PPEs in a safe manner. Such filtering techniques may also be used with existing forms of PPE.

The details of one or more implementations of the present disclosure are set forth in the accompanying drawings and the description below. Other features and advantages of the present disclosure will be apparent from the description and drawings, and from the claims.

### BRIEF DESCRIPTION OF THE DRAWINGS

FIG. 1A depicts a personal protective device with a face cover having an airflow channel and UV light elements disposed in the airflow channel.

FIG. 1B depicts a UV light element disposed in the airflow channel of FIG. 1A.

FIG. 2 depicts an airflow channel with UV light sources disposed around a perimeter of the airflow channel.

FIG. 3 depicts an airflow channel with a clear area along a portion of the perimeter of the airflow channel and a UV light source arranged to emit UV light into the airflow channel through the clear area.

FIG. 4 depicts a personal protective device having a full respirator form factor including a face cover, an inhalation element, and an exhalation element.

FIG. 5 depicts an airflow channel that includes UV lights.

FIG. 6 depicts an inhalation element with multiple airflow channels.

FIG. 7 depicts a flowchart of an example process of manufacturing a personal protective device according to the present disclosure.

### DETAILED DESCRIPTION

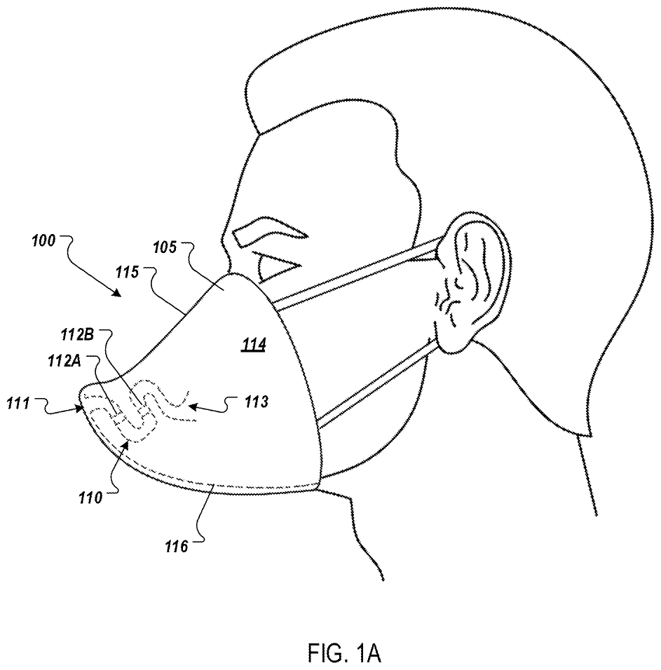

FIG. 1A depicts a personal protective device 100 with a face cover 105 having an airflow channel 110 and UV light elements 112A and 112B disposed in the airflow channel 110. FIG. 1B depicts a UV light element 112 disposed in the airflow channel 110 of FIG. 1A. The personal protective device 100 can be in the form of a face mask or respirator.

The face cover 105 can include one or more layers of material arranged to cover a portion of a person's face, e.g., to cover at least the nose and mouth of the wearer. For example, the face cover 105 can include a piece of material 114 that is folded along a fold line 115. Opposing edges of the material 114 may be closed by a seam 116 (e.g., by fusing or bonding) to form an approximately conical shape that covers the wearer's nose, mouth, and chin. In some instances, the face cover 105 may also include a substantially semispherical shell that covers the wearer's nose, mouth, and chin. The size of the face cover 105 can vary for persons of different sizes. For example, the face cover 105 can be manufactured in different sizes to fit tightly around the faces of people of different sizes. The shape of the face cover can be any variety of conical or semispherical shapes. The face cover can also have a curved edge for greater contact with the user's face.

In some implementations, the one or more layers of material can be selected to prevent air from flowing between an internal side of the face cover 105 adjacent to the person's face and an external side of the face cover 105 exposed to the environment around the person. In this example, the only airflow between the two sides of the face cover would be through the airflow channel 110, which provides a path for air to flow between the two sides of the face cover 105. Examples of such materials can include thin layers of plastics, e.g., polyethylene (PE) films and nylon films.

In some implementations, the one or more layers of material are selected to filter air traveling through the one or more layers, but not to completely block air flow. For example, the one or more layers of material can include cloth materials or multiple layers of spun-bonded polypropylene and cellulose/polyester filter membranes, similar to some N95 respirators. In some instances, an example respirator includes four layers of material: a first outer layer of spun-bond polypropylene, a second layer of cellulose of polyester, a third layer of melt-blown polypropylene filter material, and a fourth inner layer of spun-bound polypropylene, e.g., such that the second and third layers are disposed between the first and fourth layers.

In some instances, the one or more layers of material can include layers of material that are selected to block UV light. For example, the one or more layers of material can include polycarbonate UV400 films, acrylic plastics, poly (methyl methacrylate) (PMMA) films that incorporate TiO₂ and ZnO, or opaque or colored synthetic fabrics to name a few examples. The one or more UV-blocking layers can be combined with one or more layers of material that are selected to filter air or block airflow, respectively.

The airflow channel 110 enables air to flow between an inlet 111 on the external side of the face cover 105 and an outlet 113 on the internal side of the face cover 105. In some instances, the airflow channel 110 may be integrally formed with the face cover 105 (i.e., made of the same material that forms the face cover 105). For example, the seam 116 that connects the two material surfaces 114 may also form the airflow channel 110. As shown in FIG. 1A, the seam 116 may include a first section and a second section. The first section of the seam 116 may form a top edge of the airflow channel 110, and the second section of the seam 116 may form the bottom edge of the airflow channel 110 and seal the open edge of the material surface 114. The first and second sections of the seam 116 are arranged to form gaps that provide the inlet 111 and the outlet 113 of the airflow channel 110. Although the airflow channel 110 in FIG. 1A is formed by two seam sections, other implementations may include more seam sections.

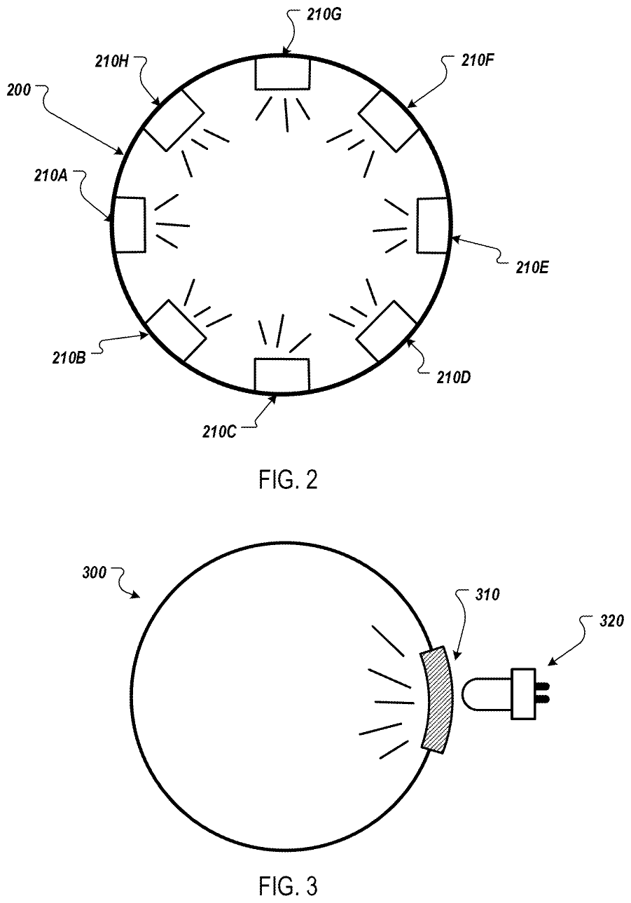

In some instances, the airflow channel 110 may be formed by a separate part that is integrated into the face cover 105. For example, the airflow channel 110 may be formed by a separate element, such as a tube, that is attached (e.g., by heat bonding, adhesive, or clips) to the inside or outside of the face cover 105 (see also FIGS. 3, 5, and 6). In such an example, the face cover 105 may include an opening that is aligned with the inlet 111 of the airflow channel 110 formed by the separate part, such that air can flow into the airflow channel 110.

Only one airflow channel 110 is illustrated in FIG. 1A. However, the personal protective device 100 can include multiple airflow channels having the same, similar, or different configurations. The quantity and arrangement of the airflow channels can be selected based on the desired amount of air flow and the size and configuration of the face cover 105. Various configurations of airflow channels are depicted in FIGS. 1B, 2, 3, 5, and 6.

In this example, the airflow channel 110 includes a meandering path between the inlet 111 and the outlet 113 and UV light elements 112A and 112B disposed in the airflow channel 110. In the context of this disclosure, “meandering path” means that the airflow channel 110 includes two or more curves or bends. For example, starting at the inlet 111, the airflow channel 110 includes six bends before arriving at the outlet 113. Other examples may include greater or fewer bends than shown in FIG. 1A. In other examples, the airflow channels can be in other shapes, e.g., straight.

The UV light elements 112A and 112B each include one or more UV light sources, such as UV light emitting diodes (LEDs). Each UV light source can emit UV light, e.g., far-UVC (207-222 nm) light that can inactivate or kill airborne particles passing through the airflow channel 110. For example, as shown in FIG. 1B, a UV light element 112 includes a UV LED 123. Although two UV light elements 112A and 112B are illustrated in FIG. 1A, other quantities of UV light elements can be disposed in the airflow channel, e.g., one, three, ten, etc.

Referring to FIG. 1B, the UV light element 112, which can be used to implement the UV light elements 112A and/or 112B in FIG. 1A, includes a UV LED 123 housed in a cone-shaped mount 122 that is held in place in the airflow channel 110 using a web-shaped holder 121. The web-shaped holder 121 can be attached to the interior walls of the airflow channel 110, e.g., using adhesives, to keep the UV light element 112 in place. The web-shaped holder 121 includes spaces between the webs to allow air to flow past the UV light element 112. Other types of holders that allow air to past the UV light element 112 while holding the UV light element 112 in place can also be used, e.g., net or lattice-shaped holders or any configuration of support struts extending from the interior walls of the airflow channel to the LED housing. In addition, other types of mounts can be used in place of the cone-shaped mount 122, such as flat or round mounts. One or more batteries can be mounted on, or in, the face cover 105 to provide power to each UV LED 123.

In this example, the UV LED 123 emits UV light in a direction opposite of the air flow within the airflow channel 110, schematically depicted by the arrows in FIG. 1B. For example, if the airflow channel 110 enables air to flow into the face cover from the external side to the internal side, the UV LED 123 is arranged to emit UV light towards the inlet 111 of the airflow channel 110. In other implementations, the UV LED 123 can be arranged to emit UV light in the direction of air flow in the airflow channel 110, e.g., by reversing the direction of the UV light element 112 in the airflow channel 110. In some instances, the airflow channel 110 can be configured to enable airflow in both directions.

The UV light element 112 can include or be disposed adjacent to or near (e.g., within a threshold distance) a filter 130. The filter 130 can include one or more layers of material for filtering pathogens and other non-pathogenic contaminants, such as smoke, particulate matter (PM), etc., from entering the mouth and nose of the wearer. For example, the filter 130 can include one or more layers of spun-bonded polypropylene and cellulose/polyester filter membranes. The filter 130 can also prevent UV light from reaching the wearer, e.g., by being made of or including an opaque material.

In this example, the filter 130 is disposed behind the UV LED 123 such that the UV LED does not emit UV light directly onto the filter 130. In other implementations, the filter 130 can be disposed in front of the UV LED 123 such that the UV LED emits UV light directly onto the filter 130, which enables the UV light to kill any pathogens on the surface of the filter 130. In another example, one or more filters 130 can be disposed in front of the UV LED 123 and one or more filters 130 can be disposed behind the UV LED 123.

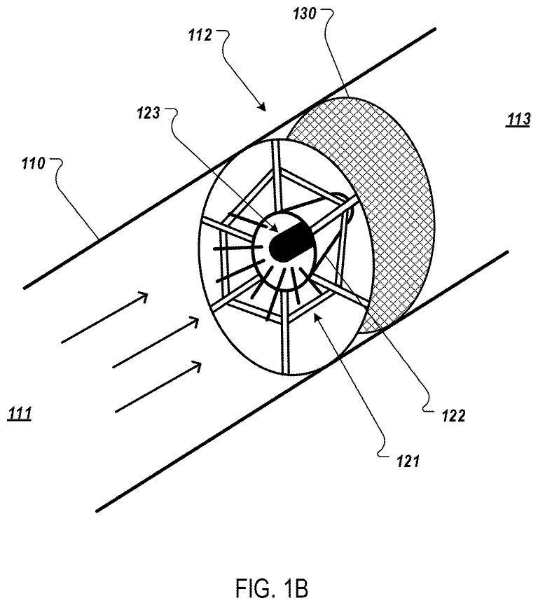

FIG. 2 depicts a cross-section through an airflow channel 200 with UV light sources 210A-210H disposed around a perimeter of the airflow channel 200. For example, the UV light sources 210A-210H may be used to collectively implement the UV light elements 112A and/or 112B shown in FIG. 1A. In this example, each UV light source 210A-210H emits UV light in a direction that is normal to the direction of air flow in the airflow channel 200. In other examples, the UV light sources 210A-210H can be arranged at an angle with respect to the wall of the airflow channel 200 to direct light in different directions, e.g., along the direction of air flow or against air flow. Although eight UV light sources 210A-210H are shown in FIG. 2, other quantities of UV light sources 210A-210H can also be used, such as one, two, five, etc. Each UV light source can be mounted on the interior surface of the airflow channel 200, e.g., using adhesives, clips, bonding, or another appropriate attachment device or technique. The airflow channel 200 can include multiple areas within the airflow channel 200 that includes one or more UV light sources mounted in this way. For example, the airflow channel 200 can include multiple rings of UV light sources that are each arranged like the UV light sources 210A-210H of FIG. 2.

The airflow channel 200 can also include one or more filters, similar to the filter 130 of FIG. 1B. The filter(s) can be located at or near, e.g., within a threshold distance along the airflow channel 200, the UV light sources 210A-210h so that the light sources 210A-210H can kill or deactivate any pathogens that collect on the surface(s) of the filter. The filter(s) can also be located elsewhere in the airflow channel 200, e.g., between sections that include LED light sources.

FIG. 3 depicts a cross-section through an airflow channel 300 with a clear area 310 along a portion of the perimeter of the airflow channel 300 and a UV light source 320 arranged to emit UV light into the airflow channel 300 through the clear area 310. For example, the airflow channel 300 can include a tube that is attached to a face cover. Other than the clear area 310, the walls of the airflow channel 300 may be opaque, e.g., to prevent the UV light from escaping the face covering. The UV light source 320 may be used to implement the UV light elements 112A and/or 112B shown in FIG. 1A. The clear area 310 can include a slit in the walls of the airflow channel. Optionally, the slit can be covered with a transparent or translucent material that enables UV light to pass from the UV light source 320 into the airflow channel 300. This configuration allows the UV light source 320 to be mounted externally to the airflow channel 300 while still emitting UV light into the airflow channel 300. For example, the UV light source 320 can be attached to the outer wall of the airflow channel 300. In some instances, the UV light source 320 can be attached (e.g., glued, mounted, or sewn) to the face cover and positioned so that one UV light source 320 can emit light through multiple clear areas 310. The external arrangement of the UV light source can reduce the drag on the flow of air caused by UV light elements disposed in the airflow channel 300.

Although FIG. 3 depicts a single UV light source 320, some implementations can include multiple light sources that are each arranged to emit UV light into the airflow channel 300 through a corresponding clear area 310. Additionally, the airflow channel 300 can include multiple UV light sources 320 that are arranged along the length of the airflow channel 300.

The airflow channel 300 can also include one or more filters, similar to the filter 130 of FIG. 1B. The filter(s) can be located at or near, e.g., within a threshold distance along the airflow channel 300, the UV light source 320 so that the light source 320 can kill or deactivate any pathogens that collect on the surface(s) of the filter. The filter(s) can also be located elsewhere in the airflow channel 300, e.g., between sections that include LED light sources.

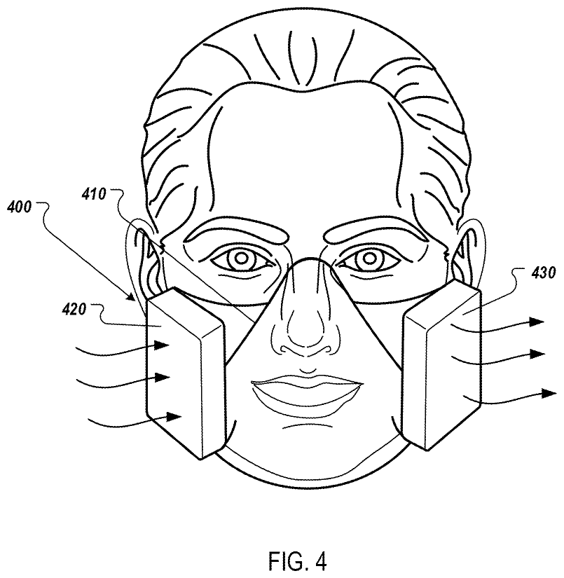

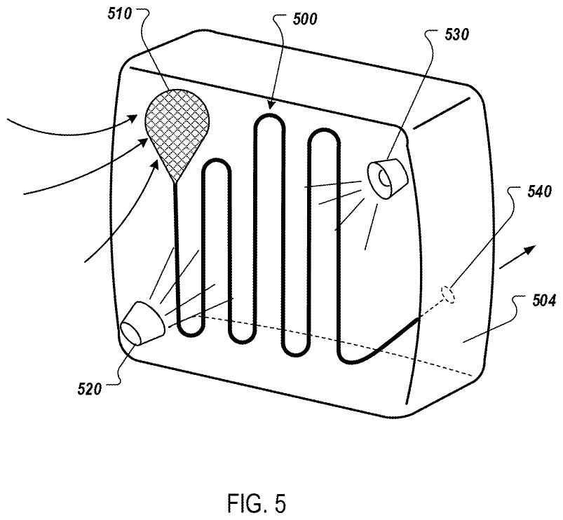

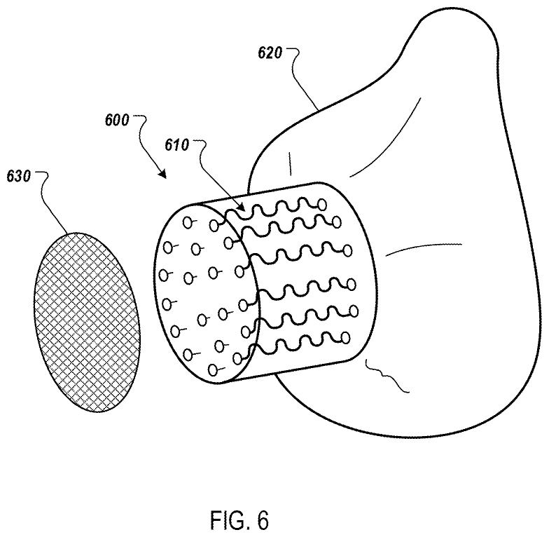

FIG. 4 depicts a personal protective device 400 having a full respirator form factor including a face cover 410, an inhalation element 420, and an exhalation element 430. The inhalation element 420 can include one or more airflow channels and one or more UV light sources that emit UV light into the airflow channel(s) to kill airborne pathogens that enter the personal protective device 400 through the inhalation element 420. Similarly, exhalation element 430 can include one or more airflow channels and one or more UV light sources that emit UV light into the airflow channel(s) to kill airborne pathogens that exit the personal protective device 400 through the exhalation element 430. Example configurations of these airflow channels are illustrated in FIGS. 5 and 6, and described below. The inhalation element 420 and/or the exhalation element 430 can also include one or more filters that filter smoke, particulate matter, etc. In some instances, the material of the face cover 410 can be selected such that the only airflow between the two sides of the face cover 410 would be through the inhalation element 420 and the exhalation element 430. In some instances, the exhalation element 430 can be replaced by an additional inhalation element 420, and a valve may be provided as a separate exhalation element.

FIG. 5 depicts an airflow channel 500 that includes an inlet 510 and an outlet 540 and is formed in a housing 504. In some instances, the airflow channel 500 can include small diameter tubing that is arranged inside the hollow housing 504. For example, the airflow channel 500 may be attached to the face cover 410 to form the inhalation element 420, as shown in FIG. 4. In this instance, the outlet 540 may communicate with the space inside of the face cover 401. The inlet 510 may be provided with a filter similar to the filter 130 in FIG. 1B. The airflow channel 500 can include transparent or translucent walls that allow UV light emitted by UV light sources 520, 530 to pass into the airflow channel 500 to kill airborne pathogens passing through the airflow channel 500. For example, the tubing can be made of clear or translucent material, e.g., polyvinyl chloride (PVC), polyurethane, silicone, or nylon to name a few examples. The tubing may be extruded, cast, or injection molded. One or more surfaces of the housing 504 may be opaque to reduce exposure to the UV light emitted by the UV light sources 520, 530. Although the airflow channel 500 is depicted with two UV light sources 520, 530, other examples may include a smaller or larger number of UV light sources. In this example, the airflow channel 500 has a meandering path, although other path shapes are possible.

The element depicted in FIG. 5 may also be used to implement the exhalation element 430 of FIG. 4. In this case, the outlet 540 may serve as the inlet, while the inlet 510 serves as the outlet to the area external to the face cover 410.

In some examples, the UV light sources can emit light into multiple airflow channels. For example, as shown in FIG. 6, an inhalation element or exhalation element 600 can attach to a face cover 620 and include multiple airflow channels 610. The airflow channels can have transparent or translucent walls that allow UV light from one or more externally mounted (relative to the channels) UV light sources (not shown) to pass into the airflow channels 610 and kill airborne pathogens in the airflow channels 610. One or more surfaces of the element 600 may be opaque to reduce exposure to UV light emitted by the UV light sources. Similarly to FIG. 3, a peripheral surface of the element 600 may be substantially opaque apart from one or more clear areas. UV light sources may be arranged to emit UV light through the one or more clear areas and onto the airflow channels 610. Similarly to FIG. 5, one or more UV light sources may be contained within a housing of the element 600 and configured to emit UV light onto the airflow channels 610.

The inhalation element or exhalation element 600 can include an optional filter cap 630 that covers the inlets of the multiple airflow channels 610. In some implementations, the airflow channel 500 and/or the airflow channels 610 can be incorporated into clip on filters that can clip onto inhalation elements and/or exhalation elements of existing respirators.

The field of light of the UV light sources can be limited to minimize refraction, e.g., to allow the UV light to kill pathogens in the airflow channels while minimizing the amount of UV light that escapes the face covers and/or reaches people's skin. For example, the UV light sources can be mounted in housing with slits that enable the UV light to shine in a limited direction.

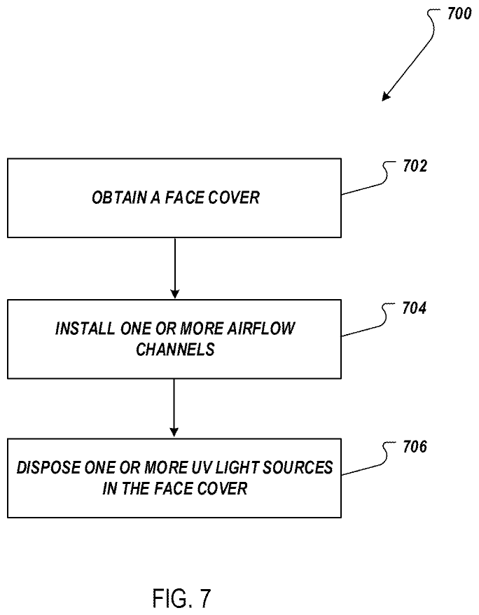

FIG. 7 is a flowchart that depicts a method 700 of manufacturing a personal protective device according to the present disclosure. The method 700 may be used to manufacture any of the personal protective devices described above. The method 700 includes obtaining 702 a face cover, installing 704 one or more airflow channels within the face cover, and disposing 706 one or more UV light sources in the face cover.

In some instances, installing 704 one or more airflow channels within the face cover may include forming the airflow channel(s) integrally with the face cover (see, e.g., FIG. 1A). In other instances, installing one or more airflow channels within the face cover may include attaching a separate element (e.g., an inhalation element or an exhalation element) to the face cover.

In some instances, disposing 706 one or more UV light sources in the face cover may include arranging the one or more UV light sources within the one or more airflow channels (see, e.g., FIGS. 1A and 1B). In other instances, disposing 706 one or more UV light sources in the face cover may include arranging the one or more UV light sources relative to a separate element that forms the one or more airflow channels and is attached to the face cover. The UV light sources may also be contained by such a separate element.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A personal protective device comprising:
a face cover arranged to cover at least a nose and mouth of a person, the face cover comprising:
one or more airflow channels that provide a path for air to flow between an exterior side of the face cover and an internal side of the face cover; and
one or more ultraviolet (UV) light sources arranged to emit UV light into the one or more airflow channels.

2. The personal protective device of claim 1, wherein the one or more UV light sources comprise one or more UV light emitting diodes (LEDs).

3. The personal protective device of claim 2, wherein the one or more UV light sources are configured to emit far-UVC light having a wavelength of about 207 to 222 nm.

4. The personal protective device of claim 1, wherein at least one UV light source is disposed within a given airflow channel and arranged to emit UV light opposite a direction of airflow within the given airflow channel.

5. The personal protective device of claim 1, wherein at least one UV light source is disposed within a given airflow channel and arranged to emit UV light in a same direction of airflow within the given airflow channel.

6. The personal protective device of claim 1, further comprising a filter disposed on at least one side of the at least one UV light source, the at least one side being either (i) in front of the at least one UV light source such that the at least one UV light source emits UV light onto the filter or (ii) behind the at least one UV light source such that the at least one UV light source emits UV light away from the filter.

7. The personal protective device of claim 1, wherein at least one airflow channel comprises a plurality of UV light sources disposed about an inner perimeter of the at least one airflow channel and arranged to emit UV light into the at least one airflow channel.

8. The personal protective device of claim 1, wherein at least one airflow channel comprises a clear area in a portion of a wall of the at least one airflow channel and a UV light source is arranged to emit UV light through the clear area and into the airflow channel.

9. The personal protective device of claim 1, wherein the face cover includes one or more layers of material that prevent air from flowing between the exterior side of the face cover and the internal side of the face cover without going through one of the one or more airflow channels.

10. The personal protective device of claim 1, wherein:
the face cover comprises a respirator comprising an inhalation element and an exhalation element; and
the one or more airflow channels are disposed within at least one of the inhalation element or the exhalation element.

11. The personal protective device of claim 1, wherein at least one airflow channel has a meandering airflow path between the exterior side of the face cover and the internal side of the face cover.

12. The personal protective device of claim 1, wherein the one or more airflow channels are integrally formed with the face cover.

13. The personal protective device of claim 1, wherein the one or more UV light sources arranged within the face cover such that, when the face cover is worn by the person, the UV light emitted by the UV light sources decontaminates air in the airflow channel and kills pathogens in the airflow channel before the air is inhaled by the person wearing the face cover.

14. A method for manufacturing a personal protective device comprising:
obtaining a face cover;
installing one or more airflow channels within the face cover; and
disposing one or more UV light sources in the face cover.

15. The method of claim 14, wherein installing one or more airflow channels within the face cover comprises forming the one or more airflow channels integrally with the face cover.

16. The method of claim 15, wherein disposing one or more UV light sources in the face cover comprises arranging the one or more UV light sources inside the one or more airflow channels.

17. The method of claim 15, wherein disposing one or more UV light sources in the face cover comprises arranging the one or more UV light sources externally to the one or more airflow channels.

18. The method of claim 14, wherein installing one or more airflow channels within the face cover comprises attaching a separate element to the face cover that forms the one or more airflow channels.

19. The method of claim 18, wherein disposing one or more UV light sources in the face cover comprises arranging the one or more UV light sources relative to a separate element.

20. The method of claim 18, wherein the one or more UV light sources are arranged within the separate element.
